(19) United States
(12) Patent Application Publication
(10) Pub. No.: US 2020/0030265 A1
(43) Pub. Date: Jan. 30, 2020

(54) ANTIPURINERGIC COMPOUNDS AND USES THEREOF

(71) Applicant: CSP PHARMA, INC., Poway, CA (US)

(72) Inventor: R. Sean PAKBAZ, Poway, CA (US)

(73) Assignee: CSP PHARMA, INC., Poway, CA (US)

(21) Appl. No.: 16/484,284

(22) PCT Filed: Feb. 7, 2018
(86) PCT No.: PCT/US2018/017200

§ 371 Date: Aug. 7, 2019

Related U.S. Application Data
(60) Provisional application No. 62/456,438, filed on Feb. 8, 2017.

(51) Int. Cl. A61K 31/185; A61P 25/00

(52) U.S. Cl. CPC A61K 31/185; A61P 25/00

(57) ABSTRACT

The disclosure herein relates to antipurinergic compounds and uses thereof. Specifically, the disclosure relates to the use of antipurinergic compounds to treat neurodevelopmental disorders. More specifically, the disclosure relates to the use of suramin for the treatment of autism spectral disorders (ASDs). More specifically, the present disclosure relates to non-intravenously administered suramin for the treatment of ASDs.

### CROSS-REFERENCE TO RELATED APPLICATION

This application claims priority to U.S. Provisional Application No. 62/456,438, filed on Feb. 8, 2017. The contents of the prior application are hereby incorporated by reference in their entirety as if set forth verbatim.

### TECHNICAL FIELD

The disclosure herein relates to antipurinergic compounds and uses thereof. Specifically, the disclosure relates to the use of antipurinergic compounds to treat neurodevelopmental disorders. More specifically, the disclosure relates to the use of suramin for the treatment of autism spectral disorders (ASDs). More specifically, the present disclosure relates to non-intravenously administered suramin for the treatment of ASDs. Non-limiting administration routes include transdermal, transmucosal, and inhalants.

### BACKGROUND

Neurodevelopmental disorders are impairment of the growth and development of the brain or central nervous system. Such disorders include autism spectral disorders (ASDs), attention deficit hyperactivity disorder (ADHD), Tourette's syndrome, schizophrenia, and epilepsy. The disorders may be caused by genetic and/or environmental factors.

ASDs encompass autism, Asperger syndrome, pervasive development disorder not otherwise specified (PDD-NOS), and childhood disintegrative disorder. Features of these disorders include social deficits and communication difficulties, stereotyped or repetitive behaviors and interests, sensory issues, and in some cases, cognitive delays.

Treatment of ASDs can involve educational and behavior therapy, medications, supplements, animal therapy, and/or dietary restrictions. Medication use is directed to alleviate specific symptoms associated with ASDs. For example, anti-depressants to treat anxiety or obsessive compulsive disorder, stimulants to treat ADHD, anticonvulsants to treat seizures, gastrointestinal agents to treat digestive disorders, and melatonin to treat sleep disorders. To date, no medication is approved to treat the core symptoms of ASDs. Accordingly, a need exists for a medication to treat the core symptoms of ASDs.

### SUMMARY

The following simplified summary provides a basic understanding of some aspects of the claimed subject matter. This summary is not an extensive overview, and is not intended to identify key/critical elements or to delineate the scope of the claimed subject matter. Its purpose is to present some concepts in a simplified form as a prelude to the more detailed description that is presented below.

In one embodiment, is a composition for use in treating neurodevelopmental disorders. The composition includes an antipurinergic compound and a carrier. The composition is formulated for non-intravenous administration. In another embodiment, the antipurinergic compound is suramin.

In another embodiment, the neurodevelopmental disorder may be autism spectral disorders, attention deficit hyperactivity disorder (ADHD), Tourette's syndrome, schizophrenia, or epilepsy. In this embodiment, the autism spectral disorder may be autism, Asperger's syndrome, pervasive developmental disorder not otherwise specified, or childhood disintegrative disorder. In one embodiment, the disorder is autism. In another embodiment, the disorder is ADHD. In another embodiment, the disorder is Tourette's syndrome. In another embodiment, the disorder is Asperger's syndrome. In another embodiment, the disorder is schizophrenia. In another embodiment, the disorder is epilepsy. In another embodiment, the disorder is pervasive development disorder with not otherwise specified. In another embodiment, the disorder is childhood disintegrative disorder.

In another embodiment, the non-intravenous delivery may be intranasal, inhalant, sublingual, buccal, lingual, gingival, transdermal, ocular, vaginal, urethral, rectal, or implantable devices. In one embodiment, the non-intravenous delivery is intranasal. In another embodiment, the non-intravenous delivery is inhalant. In another embodiment, the non-intravenous delivery is sublingual. In another embodiment, the non-intravenous delivery is buccal. In another embodiment, the non-intravenous delivery is lingual. In another embodiment, the non-intravenous delivery is gingival. In another embodiment, the non-intravenous delivery is transdermal. In another embodiment, the non-intravenous delivery is ocular. In another embodiment, the non-intravenous delivery is vaginal. In another embodiment, the non-intravenous delivery is urethral. In another embodiment, the non-intravenous delivery is rectal. In another embodiment, the non-intravenous delivery is implantable devices.

In another embodiment, the compositions and formulations described herein may further include stabilizers, preservatives, or penetration enhancers.

In another embodiment, the composition of any one of the preceding claims, further comprising and extended release agent.

In another embodiment, the compositions and formulations disclosed herein are used to treat a neurodevelopment disorder in a subject.

To the accomplishment of the foregoing and related ends, certain illustrative aspects are described herein in connection with the following description and the annexed drawings. These aspects are indicative, however, of but a few of the various ways in which the principles of the claimed subject matter may be employed and the claimed subject matter is intended to include all such aspects and their equivalents. Other advantages and novel features may become apparent from the following detailed description when considered in conjunction with the drawings

### DETAILED DESCRIPTION

Autism spectrum disorders (ASDs) are complex, multisystem disorders that are defined by unifying core abnormalities in the development of language, social behavior, and repetitive behaviors. Genetic, environmental, and metabolic factors can contribute to the risk of developing ASDs. Diagnosis is based on behavior and is characterized by persistent deficits in social communication and interaction across multiple contexts, as well as restricted, repetitive patterns of behavior, interests, or activities. Symptoms may include lack or social or emotional reciprocity, stereotyped and repetitive use of language or idiosyncratic language and persistent preoccupation with unusual objects.

Without wishing to be bound by any one theory, the present disclosure is premised on the belief that ASDs often present with abnormalities in puridine and pyrimadine metabolism. Specifically, ATP, ADP, UTP, and UDP act as signaling molecules outside the cell. They bind to and regulate purinergic receptors that are present on the surface of every cell in the body. ATP has been found to be a co-neurotransmitter at every type of synaptic junction studied to date. Excess extracellular ATP is an activator of innate and adaptive immunity, is a danger signal and damage-associated molecular pattern (DAMP) that is chemotactic for neutrophils, and a potent regulator of microglial activation, death, and survival. The concentration of extracellular nucleotides under normal circumstances is ultimately controlled by mitochondrial function and cellular health. Fifteen different isoforms of purinergic receptors are known that are stimulated by extracellular nucleotides. These are divided into ionotropic P2X receptors and metabotropic P2Y receptors. P2Y receptors are G-protein coupled receptors. Together, P2X and P2Y receptors are known to control a broad range of biological characteristics that have relevance to autism. These include all the known abnormalities that occur in autism. For example, purinergic signaling modulates normal synaptogenesis and brain development, the PI3K/AKT pathway, innate and adaptive immune responses, and chronic inflammation, neuroinflammation, antiviral signaling, microglial activation, neutrophil chemotaxis, autophagy, gut motility, gut permeability, taste chemosensory transduction, sensitivity to food allergens, hearing, and chronic pain syndromes.

Suramin, shown below, (molecular formula: C51H40N6023S6; CAS number: 145-63-1) is an antiprotozoal agent that traditionally has been used to treat African sleeping sickness (trypanozomiasis) and river blindness (onchocerciasis). Recently, suramin has been shown to have antineoplastic and antipurinergic activity. With respect to antineoplastic activity, suramin has been found to block the binding of various growth factors, including insulin-like growth factor I (IGF-I), epidermal growth factor (EGF), platelet-derived growth factor (PDGF), and tumor growth factor-beta (TGF-beta), to their receptors, thereby inhibiting endothelial cell proliferation and migration. It also inhibits vascular endothelial growth factor (VEGF)- and basic fibroblast growth factor (bFGF)-induced angiogenesis; retroviral reverse transcriptase; uncoupling of G-proteins from receptors; topoisomerases; cellular folate transport; and steroidogenesis. With respect to antipurinergic activity, suramin has been shown to inhibit excess purinergic signaling by acting as a competitive inhibitor of nucleotide signaling at both ionotropic purinergic (P2X) receptors, and G-protein coupled, metabotropic purinergic (P2Y) receptors.

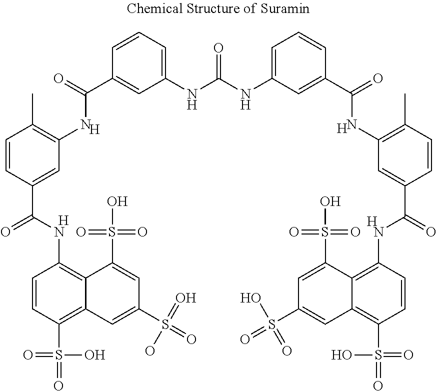

This disclosure is premised on the belief that suramin can improve social behaviors in ASDs. Specifically, surnaming was found to correct 16 multisystem abnormalities that defined the ASD-like phenotype in an ASDs mouse model. These included correction of the core social deficits and sensorimotor coordination abnormalities, prevention of cerebellar Purkinje cell loss, correction of the ultrastructural synaptic dysmorphology, and correction of the hypothermia, metabolic, mitochondrial, P2Y2 and P2X7 purinergic receptor expression, and ERK1/2 and CAMKII signal transduction abnormalities. (Naviaux, et al., PLoS One. 2013;8(3):e57380, Epub 2013 Mar. 13.)

Current formulations of suramin are not practical for treating individuals diagnosed with ASDs. Accordingly, new formulations are needed that allow for non-intravenous administration of suramin. The new formulations should be quick and painless to administer and easy for sensitive children to tolerate.

From time-to-time, the present invention is described herein in terms of example environments. Description in terms of these environments is provided to allow the various features and embodiments of the invention to be portrayed in the context of an exemplary application. After reading this description, it will become apparent to one of ordinary skill in the art how the invention can be implemented in different and alternative environments.

### Definitions

In the description that follows, a number of terms are extensively utilized. The following non-limiting definitions provide a clear and consistent understanding of the specification and claims, including the exemplary scope to be given such terms. Unless defined otherwise, all technical, scientific, and medical terms used herein have the same meaning as is commonly understood by one of ordinary skill in the art to which this disclosure belongs.

As used herein, the terms “one,” “a,” or “an” are used in this disclosure, they mean “at least one” or “one or more,” unless otherwise indicated.

As used herein, the terms “invention” or “present invention” as used herein are intended to be non-limiting and are not intended to refer to any single embodiment of the particular invention but encompasses all possible embodiments as described in the specification and the claims.

As used herein, the term “and/or” may mean “and,” it may mean “or,” it may mean “exclusive-or,” it may mean “one,” it may mean “some, but not all,” it may mean “neither,” and/or it may mean “both.”

As used herein, the terms “treatment,” “treat,” or “treating” refers to a method of reducing one or more effects a disease or condition or one or more symptoms of the disease or condition.

As used herein, the terms “effective amount,” “effective dosage,” and “therapeutically effective amount” may be used interchangeably. The term effective amount refers to any amount necessary to produce a desired physiologic response. The dosage ranges for administration are those large enough to produce the desired effect in which one or more symptoms of the disease or disorder are affected. The dosage should not be so large as to cause substantial adverse side effects. Generally, the dosage will vary with the activity of the specific compound employed, the metabolic stability and length of action of that compound, the species, age, body weight, general health, sex and diet of the subject, the mode and time of administration, rate of excretion, drug combination, and severity of the particular condition

As used herein, the terms “subject,” “patient,” and “participant” are used interchangeably. As used herein, the term “patient” refers to an animal, often a mammal, and most often a human. In one embodiment, the subject is a human infant. In another embodiment, the subject is a human toddler. In another embodiment, the subject is a human child. In other embodiments, the subject is a human pre-teen. In other embodiments, the subject is a human teenager. In other embodiments, the subject is an adult.

As used herein, the abbreviations for any protective groups, amino acids and other compounds are, unless indicated otherwise, in accord with their common usage, recognized abbreviations, or the IUPAC-IUB Commission on Biochemical Nomenclature (See, Biochem. 11:942-944 (1972)).

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure, enantiomerically enriched, racemic mixture, diastereomerically pure, diastereomerically enriched, or a stereoisomeric mixture. In addition it is understood that, in any compound described herein having one or more double bond(s) generating geometrical isomers that can be defined as E or Z, each double bond may independently be E or Z a mixture thereof.

Likewise, it is understood that, in any compound described, all tautomeric forms are also intended to be included. For example all tautomers of phosphate groups are intended to be included. Furthermore, all tautomers of heterocyclic bases known in the art are intended to be included, including tautomers of natural and non-natural purine-bases and pyrimidine-bases.

Unless otherwise defined, all terms (including technical and scientific terms) are to be given their ordinary and customary meaning to a person of ordinary skill in the art, and are not to be limited to a special or customized meaning unless expressly so defined herein. It should be noted that the use of particular terminology when describing certain features or aspects of the disclosure should not be taken to imply that the terminology is being re-defined herein to be restricted to include any specific characteristics of the features or aspects of the disclosure with which that terminology is associated. Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular embodiment of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

Where a range of values is provided, it is understood that the upper and lower limit, and each intervening value between the upper and lower limit of the range is encompassed within the embodiments.

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

It is understood that the compounds described herein can be labeled isotopically. Substitution with isotopes such as deuterium may afford certain therapeutic advantages resulting from greater metabolic stability, such as, for example, increased in vivo half-life or reduced dosage requirements. Each chemical element in a compound may be any isotope of said element. Thus, reference herein to a compound encompasses all potential isotopic forms unless the context clearly dictates otherwise.

It is understood that the methods and combinations described herein include crystalline forms (also known as polymorphs, which include the different crystal packing arrangements of the same elemental composition of a compound), amorphous phases, salts, solvates, and hydrates. In some embodiments, the compounds described herein exist in solvated forms with pharmaceutically acceptable solvents such as water, ethanol, or the like. In other embodiments, the compounds described herein exist in unsolvated form. Solvates contain either stoichiometric or non-stoichiometric amounts of a solvent, and may be formed during the process of crystallization with pharmaceutically acceptable solvents such as water, ethanol, or the like. Hydrates are formed when the solvent is water, or alcoholates are formed when the solvent is alcohol. In addition, the compounds provided herein can exist in unsolvated as well as solvated forms. In general, the solvated forms are considered equivalent to the unsolvated forms for the purposes of the compounds and methods provided herein.

The term “salt” as used herein is a broad term and includes without limitation pharmaceutically acceptable salts such as a salt of a compound that does not cause significant harm to an organism to which it is administered and does not abrogate the biological activity and properties of the compound. In some embodiments, the salt is an acid addition salt of the compound. Pharmaceutical salts can be obtained by reacting a compound with an inorganic acid, an organic acid, or a base. Suitable pharmaceutically acceptable salts include metallic salts, organic salts, salts of free acids and bases, inorganic salts, and other salts which are currently in widespread pharmaceutical use and are listed in sources well known to those of skill in the art, such as, for example, The Merck Index.

### Compositions and Formulations

To date, suramin is only available via intravenous administration. Additionally, Intramuscular and subcutaneous administration can result in local tissue inflammation or necrosis. Furthermore, it is poorly absorbed from the gastrointestinal tract. Intravenous administration is challenging in children and even more challenging if not impossible in children with ASDs. Intravenous administration on a regular basis over a lifetime is not acceptable for children with ASDs. Accordingly, new formulations that allow different delivery methods are needed. Non-limiting delivery methods include oral, inhalant, sublingual, buccal, gingival, nasal, topical, transdermal, trans-mucosal membrane, ocular, vaginal, rectal, and implantation of a slow-release device. The compositions and formulations can also be formulated as modified release dosage forms, including delayed-, extended-, prolonged-, sustained-, pulsatile-, controlled-, accelerated-, fast-, targeted-, or programmed-release.

In one embodiment, the compositions and formulations are formulated to improve the ability of suramin to cross the blood brain barrier. Non-limiting examples include intranasal delivery, sublingual delivery, focused ultrasound, proline rich peptides, nanoparticles, liposomes, ammonium bets-cyclodextrin nanoparticles, lipid nanoparticles, micelles, and the like.

In one embodiment the compositions and formulations are formulated for intranasal administration. The intranasal formulations may be in a liquid form such as a solution, an emulsion, a suspension, drops, or in a solid form such as a powder, gel, or ointment. Nasal drug delivery can be carried out using devices including, but not limited to, intranasal inhalers, intranasal spray devices, atomizers, nasal spray bottles, unit dose containers, pumps, droppers, squeeze bottles, nebulizers, metered dose inhalers (MDI), pressurized dose inhalers, insufflators, and bi-directional devices. The nasal delivery device can be metered to administer an accurate effective dosage amount to the nasal cavity. The nasal delivery device can be for single unit delivery or multiple unit delivery.

The intranasal formulation can be formulated as aerosols with or without solvents, and formulated with or without carriers. The formulation may be a solution, or may be an aqueous emulsion with one or more surfactants. For example, an aerosol spray may be generated from pressurized container with a suitable propellant such as, dichlorodifluoromethane, trichlorofluoromethane, dichlorotetrafluoroethane, hydrocarbons, compressed air, nitrogen, carbon dioxide, or other suitable gas. The dosage unit can be determined by providing a valve to deliver a metered amount. Pump spray dispensers can dispense a metered dose or a dose having a specific particle or droplet size. As used herein, the term “aerosol” refers to a suspension of fine solid particles or liquid solution droplets in a gas. Specifically, aerosol includes a gas-borne suspension of droplets of the antipurinergic agent or suramin compositions, as may be produced in any suitable device, such as an MDI, a nebulizer, or a mist sprayer. Aerosol also includes a dry powder composition of the composition of this disclosure suspended in air or other carrier gas.

The compositions and formulations may be delivered to the nasal cavity as a powder in a form such as microspheres delivered by a nasal insufflator. The nasal insufflator may be absorbed to a solid surface, for example, a carrier. The powder or microspheres may be administered in a dry, air-dispensable form. The powder or microspheres may be stored in a container of the insufflator. Alternatively the powder or microspheres may be filled into a capsule, such as a gelatin capsule, or other single dose unit adapted for nasal administration.

The intranasal formulation can be delivered to the nasal cavity by direct placement in the nasal cavity, for example, in the form of a gel, an ointment, a nasal emulsion, a lotion, a cream, a nasal tampon, a dropper, or a bioadhesive strip. In certain embodiments, it can be desirable to prolong the residence time of the pharmaceutical composition in the nasal cavity, for example, to enhance absorption. Thus, the intranasal formulation can optionally be formulated with a bioadhesive polymer, alginic acid, a gum (e.g., xanthan gum), chitosan (e.g., highly purified cationic polysaccharide), pectin (or any carbohydrate that thickens like a gel or emulsifies when applied to nasal mucosa), a microsphere (e.g., starch, albumin, dextran, cyclodextrin), gelatin, a liposome, carbamer, polyvinyl alcohol, alginate, acacia, chitosans and/or cellulose (e.g., methyl or propyl; hydroxyl or carboxy; carboxymethyl or hydroxylpropyl).

In one embodiment, the compositions and formulations are formulated using the techniques described in U.S. Pat. Nos. 8,133,863; 8,226,949; 8,268,791; 8,329,220; 8,440,631; 8,470,370; 8,551,468; 8,642,564; 8,772,231; 8,846,044; 8,883,728; 8,927,497; 9,283,280; 9,446,134 and U.S. pub. no. 20100203014 and 20160051624 all assigned to Aegis Therapeutics, LLC (San Diego, Calif.). In another embodiment, the intranasal formulations are formulated for use with a ViaNase Electronic Atomizer from Kurve Technology (Bethell, Wash.).

In another embodiment, the compositions and formulations are formulated as an inhalant. Inhalable agents include nebulizer inhalers, dry powder inhalers (DPI), and metered-dose inhalers (MDI).

Nebulizer devices produce a stream of high velocity air that causes a therapeutic agent in the form of liquid to spray as a mist. The therapeutic agent is formulated in a liquid form such as a solution or a suspension of particles of suitable size. In one embodiment, the particles are micronized. The term “micronized” is defined as having about 90% or more of the particles with a diameter of less than about 10 micrometers (“um” or “microns”). Suitable nebulizer devices are provided commercially, for example, by PARI GmbH (Starnberg, Germany). Other nebulizer devices include Respimat (Boehringer Ingelheim) and those disclosed in, for example, U.S. Pat. Nos. 7,568,480 and 6,123,068, and WO 97/12687. The inhalant formulation can be formulated for use in a nebulizer device as an aqueous solution or as a liquid suspension.

DPI devices typically administer a therapeutic agent in the form of a free flowing powder that can be dispersed in a patient's air-stream during inspiration. DPI devices which use an external energy source may also be used in the present invention. In order to achieve a free flowing powder, the therapeutic agent can be formulated with a suitable excipient (e.g., lactose). A dry powder formulation can be made, for example, by combining dry lactose having a particle size between about 1 um and 100 um with micronized particles of the antipurinergic agent or suramin compositions and dry blending. Alternatively, the antipurinergic agent or suramin compositions can be formulated without excipients. The formulation is loaded into a dry powder dispenser, or into inhalation cartridges or capsules for use with a dry powder delivery device. Examples of DPI devices provided commercially include Diskhaler (GlaxoSmithKline, Research Triangle Park, N.C.) (see, e.g., U.S. Pat. No. 5,035,237); Diskus (GlaxoSmithKline) (see, e.g., U.S. Pat. No. 6,378,519; Turbuhaler (AstraZeneca, Wilmington, Del.) (see, e.g., U.S. Pat. No. 4,524,769); and Rotahaler (GlaxoSmithKline) (see, e.g., U.S. Pat. No. 4,353,365). Further examples of suitable DPI devices are described in U.S. Pat. Nos. 5,415,162, 5,239,993, and 5,715,810 and references therein.

MDI devices typically discharge a measured amount of therapeutic agent using compressed propellant gas. Formulations for MDI administration include a solution or suspension of active ingredient in a liquefied propellant. Examples of propellants include hydrofluoroalklanes (HFA), such as 1,1,1,2-tetrafluoroethane (HFA 134a) and 1,1,1,2,3,3,3-heptafluoro-n-propane, (HFA 227), and chlorofluorocarbons, such as CC1.sub.3F. Additional components of HFA formulations for MDI administration include co-solvents, such as ethanol, pentane, water; and surfactants, such as sorbitan trioleate, oleic acid, lecithin, and glycerin. (See, for example, U.S. Pat. No. 5,225,183, EP 0717987, and WO 92/22286). The formulation is loaded into an aerosol canister, which forms a portion of an MDI device. Examples of MDI devices developed specifically for use with HFA propellants are provided in U.S. Pat. Nos. 6,006,745 and 6,143,227. For examples of processes of preparing suitable formulations and devices suitable for inhalation dosing see U.S. Pat. Nos. 6,268,533, 5,983,956, 5,874,063, and 6,221,398, and WO 99/53901, WO 00/61108, WO 99/55319 and WO 00/30614.

The compositions and formulations may be encapsulated in liposomes or microcapsules for delivery via inhalation. A liposome is a vesicle composed of a lipid bilayer membrane and an aqueous interior. The lipid membrane may be made of phospholipids, examples of which include phosphatidylcholine such as lecithin and lysolecithin; acidic phospholipids such as phosphatidylserine and phosphatidylglycerol; and sphingophospholipids such as phosphatidylethanolamine and sphingomyelin. Alternatively, cholesterol may be added. A microcapsule is a particle coated with a coating material. For example, the coating material may consist of a mixture of a film-forming polymer, a hydrophobic plasticizer, a surface activating agent or/and a lubricant nitrogen-containing polymer. U.S. Pat. Nos. 6,313,176 and 7,563,768.

In another embodiment, the compositions and formulations are formulated for ocular administration. In this embodiment, the compositions and formulations can further include a permeation enhancer. For ocular administration, the compositions described herein can be formulated as a solution, emulsion, suspension, etc. A variety of vehicles suitable for administering compounds to the eye. Non-limiting examples include those described in U.S. Pat. Nos. 6,261,547; 6,197,934; 6,056,950; 5,800,807; 5,776,445; 5,698,219; 5,521,222; 5,403,841; 5,077,033; 4,882,150; and 4,738,851.

In another embodiment, the compositions and formulations are formulated for oral administration. As used herein, oral administration also includes buccal, lingual, gingival, and sublingual administration. Suitable oral dosage forms include, but are not limited to, tablets, fastmelts, chewable tablets, capsules, pills, strips, troches, lozenges, pastilles, cachets, pellets, medicated chewing gum, bulk powders, effervescent or non-effervescent powders or granules, oral mists, solutions, emulsions, suspensions, wafers, sprinkles, elixirs, and syrups. In addition to the active ingredient(s), the compositions and formulations can include one or more pharmaceutically acceptable carriers or excipients, including, but not limited to, binders, fillers, diluents, disintegrants, wetting agents, lubricants, glidants, coloring agents, dye-migration inhibitors, sweetening agents, flavoring agents, emulsifying agents, suspending and dispersing agents, preservatives, solvents, non-aqueous liquids, organic acids, and sources of carbon dioxide. It should be understood that many carriers and excipients may serve a plurality of functions, even within the same formulation.

Binders or granulators impart cohesiveness to a tablet to ensure the tablet remaining intact after compression. Suitable binders or granulators include, but are not limited to, starches, such as corn starch, potato starch, and pre-gelatinized starch (e.g., STARCH 1500); gelatin; sugars, such as sucrose, glucose, dextrose, molasses, and lactose; natural and synthetic gums, such as acacia, alginic acid, alginates, extract of Irish moss, panwar gum, ghatti gum, mucilage of isabgol husks, carboxymethylcellulose, methylcellulose, polyvinylpyrrolidone (PVP), Veegum, larch arabogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); microcrystalline celluloses, such as AVICEL-PH-101, AVICEL-PH-103, AVICEL RC-581, AVICEL-PH-105 (FMC Corp., Marcus Hook, Pa.); and mixtures thereof. Suitable fillers include, but are not limited to, talc, calcium carbonate, microcrystalline cellulose, powdered cellulose, dextrates, kaolin, mannitol, silicic acid, sorbitol, starch, pre-gelatinized starch, and mixtures thereof. The amount of a binder or filler in the pharmaceutical composition provided herein varies upon the type of formulation, and is readily discernible to those of ordinary skill in the art. The binder or filler may be present from about 50 to about 99% by weight in the pharmaceutical composition provided herein.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets. The amount of a diluent in the pharmaceutical composition provided herein varies upon the type of formulation, and is readily discernible to those of ordinary skill in the art.

Suitable disintegrants include, but are not limited to, agar; bentonite; celluloses, such as methylcellulose and carboxymethylcellulose; wood products; natural sponge; cation-exchange resins; alginic acid; gums, such as guar gum and Veegum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; aligns; and mixtures thereof. The amount of a disintegrant in the pharmaceutical composition provided herein varies upon the type of formulation, and is readily discernible to those of ordinary skill in the art. The amount of a disintegrant in the pharmaceutical composition provided herein varies upon the type of formulation, and is readily discernible to those of ordinary skill in the art. The pharmaceutical composition provided herein may contain from about 0.5 to about 15% or from about 1 to about 5% by weight of a disintegrant.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, including peanut oil, cottonseed oil, sunflower oil, sesame oil, olive oil, corn oil, and soybean oil; zinc stearate; ethyl oleate; ethyl laureate; agar; starch; lycopodium; silica or silica gels, such as AEROSIL® 200 (W.R. Grace Co., Baltimore, Md.) and CAB-O-SIL® (Cabot Co. of Boston, Mass.); and mixtures thereof. The pharmaceutical composition provided herein may contain about 0.1 to about 5% by weight of a lubricant.

Suitable glidants include, but are not limited to, colloidal silicon dioxide, CAB-O-SIL® (Cabot Co. of Boston, Mass.), and asbestos-free talc. Suitable coloring agents include, but are not limited to, any of the approved, certified, water soluble FD&C dyes, and water insoluble FD&C dyes suspended on alumina hydrate, and color lakes and mixtures thereof. A color lake is the combination by adsorption of a water-soluble dye to a hydrous oxide of a heavy metal, resulting in an insoluble form of the dye. Suitable flavoring agents include, but are not limited to, natural flavors extracted from plants, such as fruits, and synthetic blends of compounds which produce a pleasant taste sensation, such as peppermint and methyl salicylate. Suitable sweetening agents include, but are not limited to, sucrose, lactose, mannitol, syrups, glycerin, and artificial sweeteners, such as saccharin and aspartame. Suitable emulsifying agents include, but are not limited to, gelatin, acacia, tragacanth, bentonite, and surfactants, such as polyoxyethylene sorbitan monooleate (TWEEN® 20), polyoxyethylene sorbitan monooleate 80 (TWEEN® 80), and triethanolamine oleate. Suitable suspending and dispersing agents include, but are not limited to, sodium carboxymethylcellulose, pectin, tragacanth, Veegum, acacia, sodium carbomethylcellulose, hydroxypropyl methylcellulose, and polyvinylpyrrolidone. Suitable preservatives include, but are not limited to, glycerin, methyl and propylparaben, benzoic add, sodium benzoate and alcohol. Suitable wetting agents include, but are not limited to, propylene glycol monostearate, sorbitan monooleate, diethylene glycol monolaurate, and polyoxyethylene lauryl ether. Suitable solvents include, but are not limited to, glycerin, sorbitol, ethyl alcohol, and syrup. Suitable non-aqueous liquids utilized in emulsions include, but are not limited to, mineral oil and cottonseed oil. Suitable organic acids include, but are not limited to, citric and tartaric acid. Suitable sources of carbon dioxide include, but are not limited to, sodium bicarbonate and sodium carbonate.

The compositions and formulations provided herein for oral administration can be provided as compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenyl salicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

The tablet dosage forms can be prepared from the active ingredient in powdered, crystalline, or granular forms, alone or in combination with one or more carriers or excipients described herein, including binders, disintegrants, controlled-release polymers, lubricants, diluents, and/or colorants. Flavoring and sweetening agents are especially useful in the formation of chewable tablets and lozenges.

The compositions and formulations provided herein for oral administration can be provided as soft or hard capsules, which can be made from gelatin, methylcellulose, starch, or calcium alginate. The hard gelatin capsule, also known as the dry-filled capsule (DFC), consists of two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The liquid, semisolid, and solid dosage forms provided herein may be encapsulated in a capsule. Suitable liquid and semisolid dosage forms include solutions and suspensions in propylene carbonate, vegetable oils, or triglycerides. Capsules containing such solutions can be prepared as described in U.S. Pat. Nos. 4,328,245; 4,409,239; and 4,410,545. The capsules may also be coated as known by those of skill in the art in order to modify or sustain dissolution of the active ingredient.

The compositions and formulations provided herein for oral administration can be provided in liquid and semisolid dosage forms, including emulsions, solutions, suspensions, elixirs, and syrups. An emulsion is a two-phase system, in which one liquid is dispersed in the form of small globules throughout another liquid, which can be oil-in-water or water-in-oil. Emulsions may include a pharmaceutically acceptable non-aqueous liquid or solvent, emulsifying agent, and preservative. Suspensions may include a pharmaceutically acceptable suspending agent and preservative. Aqueous alcoholic solutions may include a pharmaceutically acceptable acetal, such as a di(lower alkyl) acetal of a lower alkyl aldehyde, e.g., acetaldehyde diethyl acetal; and a water-miscible solvent having one or more hydroxyl groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

Other useful liquid and semisolid dosage forms include, but are not limited to, those containing the active ingredient(s) provided herein, and a dialkylated mono- or poly-alkylene glycol, including, 1,2-dimethoxymethane, diglyme, triglyme, tetraglyme, polyethylene glycol-350-dimethyl ether, polyethylene glycol-550-dimethyl ether, polyethylene glycol-750-dimethyl ether, wherein 350, 550, and 750 refer to the approximate average molecular weight of the polyethylene glycol. These formulations can further comprise one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, bisulfite, sodium metabisulfite, thiodipropionic acid and its esters, and dithiocarbamates.

The compositions and formulations provided herein for oral administration can be also provided in the forms of liposomes, micelles, microspheres, or nanosystems. Micellar dosage forms can be prepared as described in U.S. Pat. No. 6,350,458.

The composition and formulations provided herein for oral administration can be provided as non-effervescent or effervescent, granules and powders, to be reconstituted into a liquid dosage form. Pharmaceutically acceptable carriers and excipients used in the non-effervescent granules or powders may include diluents, sweeteners, and wetting agents. Pharmaceutically acceptable carriers and excipients used in the effervescent granules or powders may include organic acids and a source of carbon dioxide.

Coloring and flavoring agents can be used in all of the above compositions and formulations.

The compositions and formulations provided herein for oral administration can be formulated as immediate or modified release dosage forms, including delayed-, sustained, pulsed-, controlled, targeted-, and programmed-release forms.

In another embodiment, the compositions and formulations are formulated for topical administration to skin, orifices, or mucosa. Topical administration, as used herein, includes (intra)dermal, conjunctival, intracorneal, intraocular, ophthalmic, auricular, transdermal, nasal, vaginal, urethral, respiratory, and rectal administration.

The compositions and formulations provided herein can be formulated in any dosage forms that are suitable for topical administration for local or systemic effect, including emulsions, solutions, suspensions, creams, gels, hydrogels, ointments, dusting powders, dressings, elixirs, lotions, suspensions, tinctures, pastes, foams, films, aerosols, irrigations, sprays, suppositories, bandages, and dermal patches. The topical formulation of the pharmaceutical composition provided herein can also comprise liposomes, micelles, microspheres, nanosystems, and mixtures thereof.

Pharmaceutically acceptable carriers and excipients suitable for use in the topical formulations provided herein include, but are not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, penetration enhancers, cryoprotectants, lyoprotectants, thickening agents, and inert gases.

The compositions and formulations can also be administered topically by electroporation, iontophoresis, phonophoresis, sonophoresis, or microneedle or needle-free injection, such as POWDERJECT™ (Chiron Corp., Emeryville, Calif.), and BIOJECT™ (Bioject Medical Technologies Inc., Tualatin, Oreg.).

The compositions and formulations provided herein can be provided in the forms of ointments, creams, and gels. Suitable ointment vehicles include oleaginous or hydrocarbon vehicles, including lard, benzoinated lard, olive oil, cottonseed oil, and other oils, white petrolatum; emulsifiable or absorption vehicles, such as hydrophilic petrolatum, hydroxystearin sulfate, and anhydrous lanolin; water-removable vehicles, such as hydrophilic ointment; water-soluble ointment vehicles, including polyethylene glycols of varying molecular weight; emulsion vehicles, either water-in-oil (W/O) emulsions or oil-in-water (O/W) emulsions, including cetyl alcohol, glyceryl monostearate, lanolin, and stearic acid. These vehicles are emollient but generally require addition of antioxidants and preservatives.

Suitable cream base can be oil-in-water or water-in-oil. Suitable cream vehicles may be water-washable, and contain an oil phase, an emulsifier, and an aqueous phase. The oil phase is also called the “internal” phase, which is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation may be a nonionic, anionic, cationic, or amphoteric surfactant.

Gels are semisolid, suspension-type systems. Single-phase gels contain organic macromolecules distributed substantially uniformly throughout the liquid carrier. Suitable gelling agents include, but are not limited to, crosslinked acrylic acid polymers, such as carbomers, carboxypolyalkylenes, and CARBOPOL®; hydrophilic polymers, such as polyethylene oxides, polyoxyethylene-polyoxypropylene copolymers, and polyvinylalcohol; cellulosic polymers, such as hydroxypropyl cellulose, hydroxyethyl cellulose, hydroxypropyl methylcellulose, hydroxypropyl methylcellulose phthalate, and methylcellulose; gums, such as tragacanth and xanthan gum; sodium alginate; and gelatin. In order to prepare a uniform gel, dispersing agents such as alcohol or glycerin can be added, or the gelling agent can be dispersed by trituration, mechanical mixing, and/or stirring.

The compositions and formulations provided herein can be administered rectally, urethrally, vaginally, or perivaginally in the forms of suppositories, pessaries, bougies, poultices or cataplasm, pastes, powders, dressings, creams, plasters, contraceptives, ointments, solutions, emulsions, suspensions, tampons, gels, foams, sprays, or enemas.

Rectal, urethral, and vaginal suppositories are solid bodies for insertion into body orifices, which are solid at ordinary temperatures but melt or soften at body temperature to release the active ingredient(s) inside the orifices. Pharmaceutically acceptable carriers utilized in rectal and vaginal suppositories include bases or vehicles, such as stiffening agents, which produce a melting point in the proximity of body temperature, when formulated with the pharmaceutical composition provided herein; and antioxidants as described herein, including bisulfite and sodium metabisulfite. Suitable vehicles include, but are not limited to, cocoa butter (theobroma oil), glycerin-gelatin, carbowax (polyoxyethylene glycol), spermaceti, paraffin, white and yellow wax, and appropriate mixtures of mono-, di- and triglycerides of fatty acids, and hydrogels, such as polyvinyl alcohol, hydroxyethyl methacrylate, and polyacrylic acid;. Combinations of the various vehicles can also be used. Rectal and vaginal suppositories may be prepared by compressing or molding. The typical weight of a rectal and vaginal suppository is about 2 to about 3 g.

The compositions and formulations provided herein can be administered ophthalmically in the forms of solutions, suspensions, ointments, emulsions, gel-forming solutions, powders for solutions, gels, ocular inserts, and implants.

The composition and formulations provided herein can be administered intranasally or by inhalation to the respiratory tract. The pharmaceutical composition can be provided in the form of an aerosol or solution for delivery using a pressurized container, pump, spray, atomizer, such as an atomizer using electrohydrodynamics to produce a fine mist, or nebulizer, alone or in combination with a suitable propellant, such as 1,1,1,2-tetrafluoroethane or 1,1,1,2,3,3,3-heptafluoropropane. The pharmaceutical composition can also be provided as a dry powder for insufflation, alone or in combination with an inert carrier such as lactose or phospholipids; and nasal drops. For intranasal use, the powder can comprise a bioadhesive agent, including chitosan or cyclodextrin.

Solutions or suspensions for use in a pressurized container, pump, spray, atomizer, or nebulizer can be formulated to contain ethanol, aqueous ethanol, or a suitable alternative agent for dispersing, solubilizing, or extending release of the active ingredient provided herein; a propellant as solvent; and/or a surfactant, such as sorbitan trioleate, oleic acid, or an oligolactic acid.

The compositions and formulations provided herein can be micronized to a size suitable for delivery by inhalation, such as about 50 micrometers or less, or about 10 micrometers or less. Particles of such sizes can be prepared using a comminuting method such as spiral jet milling, fluid bed jet milling, supercritical fluid processing to form nanoparticles, high pressure homogenization, or spray drying.

Capsules, blisters, and cartridges for use in an inhaler or insufflator can be formulated to contain a powder mix of the pharmaceutical composition provided herein; a suitable powder base, such as lactose or starch; and a performance modifier, such as l/-leucine, mannitol, or magnesium stearate. The lactose may be anhydrous or in the form of the monohydrate. Other suitable excipients or carriers include, but are not limited to, dextran, glucose, maltose, sorbitol, xylitol, fructose, sucrose, and trehalose. The compositions and formulations provided herein for inhaled/intranasal administration can further comprise a suitable flavor, such as menthol and levomenthol; and/or sweeteners, such as saccharin and saccharin sodium.

The composition and formulations provided herein for topical administration can be formulated to be immediate release or modified release, including delayed-, sustained-, pulsed-, controlled-, targeted, and programmed release.

In another embodiment, the compositions and formulations are formulated for parenteral administration. Parenteral administration, as used herein, include intravenous, intraarterial, intraperitoneal, intrathecal, intraventricular, intraurethral, intrasternal, intracranial, intramuscular, intrasynovial, intravesical, implantation, and subcutaneous administration. In some embodiments, the parenteral administration uses a pump.

The composition and formulations provided herein for parenteral administration can be formulated in any dosage forms that are suitable for parenteral administration, including solutions, suspensions, emulsions, micelles, liposomes, microspheres, nanosystems, and solid forms suitable for solutions or suspensions in liquid prior to injection.

The compositions and formulations intended for parenteral administration can include one or more pharmaceutically acceptable carriers and excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

Suitable aqueous vehicles include, but are not limited to, water, saline, physiological saline or phosphate buffered saline (PBS), sodium chloride injection, Ringers injection, isotonic dextrose injection, sterile water injection, dextrose and lactated Ringers injection. Suitable non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, and palm seed oil. Suitable water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, N,N-dimethylacetamide, and dimethyl sulfoxide.

Suitable antimicrobial agents or preservatives include, but are not limited to, phenols, cresols, mercurials, benzyl alcohol, chlorobutanol, methyl and propyl p-hydroxybenzoates, thimerosal, benzalkonium chloride (e.g., benzethonium chloride), methyl- and propyl-parabens, and sorbic acid. Suitable isotonic agents include, but are not limited to, sodium chloride, glycerin, and dextrose. Suitable buffering agents include, but are not limited to, phosphate and citrate. Suitable antioxidants are those as described herein, including bisulfite and sodium metabisulfite. Suitable local anesthetics include, but are not limited to, procaine hydrochloride. Suitable suspending and dispersing agents are those as described herein, including sodium carboxymethylcelluose, hydroxypropyl methylcellulose, and polyvinylpyrrolidone. Suitable emulsifying agents are those described herein, including polyoxyethylene sorbitan monolaurate, polyoxyethylene sorbitan monooleate 80, and triethanolamine oleate. Suitable sequestering or chelating agents include, but are not limited to EDTA. Suitable pH adjusting agents include, but are not limited to, sodium hydroxide, hydrochloric acid, citric acid, and lactic acid. Suitable complexing agents include, but are not limited to, cyclodextrins, including α-cyclodextrin, β-cyclodextrin, hydroxypropyl-β-cyclodextrin, sulfobutylether-β-cyclodextrin, and sulfobutylether 7-β-cyclodextrin (CAPTISOL®, CyDex, Lenexa, Kans.).

When the compositions and formulations provided herein is formulated for multiple dosage administration, the multiple dosage parenteral formulations must contain an antimicrobial agent at bacteriostatic or fungistatic concentrations. All parenteral compositions and formulations must be sterile, as known and practiced in the art.

In one embodiment, the compositions and formulations for parenteral administration are provided as ready-to-use sterile solutions. In another embodiment, the compositions and formulations are provided as sterile dry soluble products, including lyophilized powders and hypodermic tablets, to be reconstituted with a vehicle prior to use. In yet another embodiment, the compositions and formulations are provided as ready-to-use sterile suspensions. In yet another embodiment, the composition and formulations are provided as sterile dry insoluble products to be reconstituted with a vehicle prior to use. In still another embodiment, the compositions and formulations are provided as ready-to-use sterile emulsions.

The compositions and formulations provided herein for parenteral administration can be formulated as immediate or modified release dosage forms, including delayed-, sustained, pulsed-, controlled, targeted-, and programmed-release forms.

The compositions and formulations provided herein for parenteral administration can be formulated as a suspension, solid, semi-solid, or thixotropic liquid, for administration as an implanted depot. In one embodiment, the pharmaceutical composition provided herein is dispersed in a solid inner matrix, which is surrounded by an outer polymeric membrane that is insoluble in body fluids but allows the active ingredient in the pharmaceutical composition diffuse through.

Suitable inner matrixes include, but are not limited to, polymethylmethacrylate, polybutyl-methacrylate, plasticized or unplasticized polyvinylchloride, plasticized nylon, plasticized polyethylene terephthalate, natural rubber, polyisoprene, polyisobutylene, polybutadiene, polyethylene, ethylene-vinyl acetate copolymers, silicone rubbers, polydimethylsiloxanes, silicone carbonate copolymers, hydrophilic polymers, such as hydrogels of esters of acrylic and methacrylic acid, collagen, cross-linked polyvinyl alcohol, and cross-linked partially hydrolyzed polyvinyl acetate.

Suitable outer polymeric membranes include but are not limited to, polyethylene, polypropylene, ethylene/propylene copolymers, ethylene/ethyl acrylate copolymers, ethylene/vinyl acetate copolymers, silicone rubbers, polydimethyl siloxanes, neoprene rubber, chlorinated polyethylene, polyvinylchloride, vinyl chloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer.

The compositions and formulations provided herein can be formulated for use with an implantable device. Such devices include implantable pumps (e.g. infusion, peristaltic, osmotic, and positive displacement), non-degradable implants, biodegradable implants, and the like. The pumps may be integrated with a continuous monitoring system programmed to maintain suramin blood levels within a certain range.

The compositions and formulations provided herein can be formulated as a modified release dosage form. As used herein, the term “modified release” refers to a dosage form in which the rate or place of release of the active ingredient(s) is different from that of an immediate dosage form when administered by the same route. Modified release dosage forms include, but are not limited to, delayed-, extended-, prolonged-, sustained-, pulsatile-, controlled-, accelerated- and fast-, targeted-, programmed-release, and gastric retention dosage forms. The pharmaceutical composition in modified release dosage forms can be prepared using a variety of modified release devices and methods known to those skilled in the art, including, but not limited to, matrix controlled release devices, osmotic controlled release devices, multiparticulate controlled release devices, ion-exchange resins, enteric coatings, multilayered coatings, microspheres, liposomes, and combinations thereof. The release rate of the active ingredient(s) can also be modified by varying the particle sizes and polymorphorism of the active ingredient(s).

Examples of modified release include, but are not limited to, those described in U.S. Pat. Nos.: 3,845,770; 3,916,899; 3,536,809; 3,598,123; 4,008,719; 5,674,533; 5,059,595; 5,591,767; 5,120,548; 5,073,543; 5,639,476; 5,354,556; 5,639,480; 5,733,566; 5,739,108; 5,891,474; 5,922,356; 5,958,458; 5,972,891; 5,980,945; 5,993,855; 6,045,830; 6,087,324; 6,113,943; 6,197,350; 6,248,363; 6,264,970; 6,267,981; 6,270,798; 6,375,987; 6,376,461; 6,419,961; 6,589,548; 6,613,358; 6,623,756; 6,699,500; 6,793,936; 6,827,947; 6,902,742; 6,958,161; 7,255,876; 7,416,738; 7,427,414; 7,485,322; Bussemer et al., Crit. Rev. Ther. Drug Carrier Syst. 2001, 18, 433-458; Modified-Release Drug Delivery Technology, 2nd ed.; Rathbone et al., Eds.; Marcel Dekker AG: 2005; Maroni et al., Expert. Opin. Drug Deliv. 2005, 2, 855-871; Shi et al., Expert Opin. Drug Deliv. 2005, 2, 1039-1058; Polymers in Drug Delivery; Ijeoma et al., Eds.; CRC Press LLC: Boca Raton, Fla., 2006; Badawy et al., J. Pharm. Sci. 2007, 9, 948-959; Modified-Release Drug Delivery Technology, supra; Conway, Recent Pat. Drug Deliv. Formul. 2008, 2, 1-8; Gazzaniga et al., Eur. J. Pharm. Biopharm. 2008, 68, 11-18; Nagarwal et al., Curr. Drug Deliv. 2008, 5, 282-289; Gallardo et al., Pharm. Dev. Technol. 2008, 13, 413-423; Chrzanowski, AAPS PharmSciTech. 2008, 9, 635-638; Chrzanowski, AAPS PharmSciTech. 2008, 9, 639-645; Kalantzi et al., Recent Pat. Drug Deliv. Formul. 2009, 3, 49-63; Saigal et al., Recent Pat. Drug Deliv. Formul. 2009, 3, 64-70; and Roy et al., J. Control Release 2009, 134, 74-80.

The compositions and formulations provided herein in a modified release dosage form can be fabricated using a matrix controlled release device known to those skilled in the art. See, Takada et al. in Encyclopedia of Controlled Drug Delivery; Mathiowitz Ed.; Wiley: 1999; Vol 2.

In certain embodiments, the compositions and formulations provided herein in a modified release dosage form is formulated using an erodible matrix device, which is water-swellable, erodible, or soluble polymers, including, but not limited to, synthetic polymers, and naturally occurring polymers and derivatives, such as polysaccharides and proteins.

Materials useful in forming an erodible matrix include, but are not limited to, chitin, chitosan, dextran, and pullulan; gum agar, gum arabic, gum karaya, locust bean gum, gum tragacanth, carrageenans, gum ghatti, guar gum, xanthan gum, and scleroglucan; starches, such as dextrin and maltodextrin; hydrophilic colloids, such as pectin; phosphatides, such as lecithin; alginates; propylene glycol alginate; gelatin; collagen; cellulosics, such as ethyl cellulose (EC), methylethyl cellulose (MEC), carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose (HPMC), HPMCP, HPMCAS, hydroxypropyl methyl cellulose acetate trimellitate (HPMCAT), and ethyl hydroxyethyl cellulose (EHEC); polyvinyl pyrrolidone; polyvinyl alcohol; polyvinyl acetate; glycerol fatty acid esters; polyacrylamide; polyacrylic acid; copolymers of ethacrylic acid or methacrylic acid (EUIDRAGIT®, Rohm America, Inc., Piscataway, N.J.); poly(2-hydroxyethyl-methacrylate); polylactides; copolymers of L-glutamic acid and ethyl-L-glutamate; degradable lactic acid-glycolic acid copolymers; poly-D-(-)-3-hydroxybutyric acid; and other acrylic acid derivatives, such as homopolymers and copolymers of butylmethacrylate, methyl methacrylate, ethyl methacrylate, ethylacrylate, (2-dimethylaminoethyl)methacrylate, and (trimethylaminoethyl)methacrylate chloride.

In certain embodiments, the compositions and formulations provided herein are formulated with a non-erodible matrix device. The active ingredient(s) is dissolved or dispersed in an inert matrix and is released primarily by diffusion through the inert matrix once administered. Materials suitable for use as a non-erodible matrix device include, but are not limited to, insoluble plastics, such as polyethylene, polypropylene, polyisoprene, polyisobutylene, polybutadiene, polymethylmethacrylate, polybutylmethacrylate, chlorinated polyethylene, polyvinlchloride, methyl acrylate-methyl methacrylate copolymers, ethylene-vinyl acetate copolymers ethylene/propylene copolymers, ethylene/ethyl acrylate copolymers, vinyl chloride copolymers with vinyl acetate, vinylidene chloride, ethylene and propylene, ionomer polyethylene terephthalate, butyl rubbers, epichlorohydrin rubbers, ethylene/vinyl alcohol copolymer, ethylene/vinyl acetate/vinyl alcohol terpolymer, ethylene/vinyloxyethanol copolymer, polyvinyl chloride, plasticized nylon, plasticized polyethylene terephthalate, natural rubber, silicone rubbers, polydimethylsiloxanes, and silicone carbonate copolymers; hydrophilic polymers, such as ethyl cellulose, cellulose acetate, crospovidone, and cross-linked partially hydrolyzed polyvinyl acetate; and fatty compounds, such as carnauba wax, microcrystalline wax, and triglycerides.

In a matrix controlled release system, the desired release kinetics can be controlled, for example, via the polymer type employed, the polymer viscosity, the particle sizes of the polymer and/or the active ingredient(s), the ratio of the active ingredient(s) versus the polymer, and other excipients or carriers in the pharmaceutical composition.

The compositions and formulations provided herein in a modified release dosage form can be prepared by methods known to those skilled in the art, including direct compression, dry or wet granulation followed by compression, and melt-granulation followed by compression.

The compositions and formulations provided herein in a modified release dosage form can be fabricated using an osmotic controlled release device, including, but not limited to, one-chamber system, two-chamber system, asymmetric membrane technology (AMT), and extruding core system (ECS). In general, such devices have at least two components: (a) a core which contains an active ingredient; and (b) a semipermeable membrane with at least one delivery port, which encapsulates the core. The semipermeable membrane controls the influx of water to the core from an aqueous environment of use so as to cause drug release by extrusion through the delivery port(s).

In addition to the active ingredient(s), the core of the osmotic device optionally includes an osmotic agent, which creates a driving force for transport of water from the environment of use into the core of the device. One class of osmotic agents is water-swellable hydrophilic polymers, which are also referred to as “osmopolymers” and “hydrogels.” Suitable water-swellable hydrophilic polymers as osmotic agents include, but are not limited to, hydrophilic vinyl and acrylic polymers, polysaccharides such as calcium alginate, polyethylene oxide (PEO), polyethylene glycol (PEG), polypropylene glycol (PPG), poly(2-hydroxyethyl methacrylate), poly(acrylic) acid, poly(methacrylic) acid, polyvinylpyrrolidone (PVP), crosslinked PVP, polyvinyl alcohol (PVA), PVA/PVP copolymers, PVA/PVP copolymers with hydrophobic monomers such as methyl methacrylate and vinyl acetate, hydrophilic polyurethanes containing large PEO blocks, sodium croscarmellose, carrageenan, hydroxyethyl cellulose (HEC), hydroxypropyl cellulose (HPC), hydroxypropyl methyl cellulose (HPMC), carboxymethyl cellulose (CMC) and carboxyethyl, cellulose (CEC), sodium alginate, polycarbophil, gelatin, xanthan gum, and sodium starch glycolate.

The other class of osmotic agents is osmogens, which are capable of imbibing water to affect an osmotic pressure gradient across the barrier of the surrounding coating. Suitable osmogens include, but are not limited to, inorganic salts, such as magnesium sulfate, magnesium chloride, calcium chloride, sodium chloride, lithium chloride, potassium sulfate, potassium phosphates, sodium carbonate, sodium sulfite, lithium sulfate, potassium chloride, and sodium sulfate; sugars, such as dextrose, fructose, glucose, inositol, lactose, maltose, mannitol, raffinose, sorbitol, sucrose, trehalose, and xylitol; organic acids, such as ascorbic acid, benzoic acid, fumaric acid, citric acid, maleic acid, sebacic acid, sorbic acid, adipic acid, edetic acid, glutamic acid, p-toluenesulfonic acid, succinic acid, and tartaric acid; urea; and mixtures thereof.

Osmotic agents of different dissolution rates can be employed to influence how rapidly the active ingredient(s) is initially delivered from the dosage form. For example, amorphous sugars, such as MANNOGEM™ EZ (SPI Pharma, Lewes, Del.) can be used to provide faster delivery during the first couple of hours to promptly produce the desired therapeutic effect, and gradually and continually release of the remaining amount to maintain the desired level of therapeutic or prophylactic effect over an extended period of time. In this case, the active ingredient(s) is released at such a rate to replace the amount of the active ingredient metabolized and excreted.

The core can also include a wide variety of other excipients and carriers as described herein to enhance the performance of the dosage form or to promote stability or processing.

Materials useful in forming the semipermeable membrane include various grades of acrylics, vinyls, ethers, polyamides, polyesters, and cellulosic derivatives that are water-permeable and water-insoluble at physiologically relevant pHs, or are susceptible to being rendered water-insoluble by chemical alteration, such as crosslinking. Examples of suitable polymers useful in forming the coating, include plasticized, unplasticized, and reinforced cellulose acetate (CA), cellulose diacetate, cellulose triacetate, CA propionate, cellulose nitrate, cellulose acetate butyrate (CAB), CA ethyl carbamate, CAP, CA methyl carbamate, CA succinate, cellulose acetate trimellitate (CAT), CA dimethylaminoacetate, CA ethyl carbonate, CA chloroacetate, CA ethyl oxalate, CA methyl sulfonate, CA butyl sulfonate, CA p-toluene sulfonate, agar acetate, amylose triacetate, beta glucan acetate, beta glucan triacetate, acetaldehyde dimethyl acetate, triacetate of locust bean gum, hydroxylated ethylene-vinylacetate, EC, PEG, PPG, PEG/PPG copolymers, PVP, HEC, HPC, CMC, CMEC, HPMC, HPMCP, HPMCAS, HPMCAT, poly(acrylic) acids and esters and poly-(methacrylic) acids and esters and copolymers thereof, starch, dextran, dextrin, chitosan, collagen, gelatin, polyalkenes, polyethers, polysulfones, polyethersulfones, polystyrenes, polyvinyl halides, polyvinyl esters and ethers, natural waxes, and synthetic waxes.

Semipermeable membrane can also be a hydrophobic microporous membrane, wherein the pores are substantially filled with a gas and are not wetted by the aqueous medium but are permeable to water vapor, as disclosed in U.S. Pat. No. 5,798,119. Such hydrophobic but water-vapor permeable membrane are typically composed of hydrophobic polymers such as polyalkenes, polyethylene, polypropylene, polytetrafluoroethylene, polyacrylic acid derivatives, polyethers, polysulfones, polyethersulfones, polystyrenes, polyvinyl halides, polyvinylidene fluoride, polyvinyl esters and ethers, natural waxes, and synthetic waxes.

The delivery port(s) on the semipermeable membrane can be formed post-coating by mechanical or laser drilling. Delivery port(s) can also be formed in situ by erosion of a plug of water-soluble material or by rupture of a thinner portion of the membrane over an indentation in the core. In addition, delivery ports can be formed during coating process, as in the case of asymmetric membrane coatings of the type disclosed in U.S. Pat. Nos. 5,612,059 and 5,698,220.

The total amount of the active ingredient(s) released and the release rate can substantially by modulated via the thickness and porosity of the semipermeable membrane, the composition of the core, and the number, size, and position of the delivery ports.

The compositions and formulations in an osmotic controlled-release dosage form can further comprise additional conventional excipients or carriers as described herein to promote performance or processing of the formulation.

The osmotic controlled-release dosage forms can be prepared according to conventional methods and techniques. See, Remington: The Science and Practice of Pharmacy, supra; Santus and Baker, J. Controlled Release 1995, 35, 1-21; Verma et al., Drug Development and Industrial Pharmacy 2000, 26, 695-708; and Verma et al., J. Controlled Release 2002, 79, 7-27.

In certain embodiments, the compositions and formulations provided herein is formulated as AMT controlled-release dosage form, which comprises an asymmetric osmotic membrane that coats a core comprising the active ingredient(s) and other pharmaceutically acceptable excipients or carriers. See, U.S. Pat. No. 5,612,059 and International Pat. Appl. Publ. No. WO 2002/17918. The AMT controlled-release dosage forms can be prepared according to conventional methods and techniques known to those skilled in the art, including direct compression, dry granulation, wet granulation, and a dip-coating method.

In certain embodiments, the compositions and formulations provided herein is formulated as ESC controlled-release dosage form, which comprises an osmotic membrane that coats a core comprising the active ingredient(s), a hydroxylethyl cellulose, and other pharmaceutically acceptable excipients or carriers.

The compositions and formulations provided herein in a modified release dosage form can be fabricated as a multiparticulate controlled release device, which comprises a multiplicity of particles, granules, or pellets, ranging from about 10 μm to about 3 mm, about 50 μm to about 2.5 mm, or from about 100 μm to about 1 mm in diameter. Such multiparticulates can be made by the processes known to those skilled in the art, including wet-and dry-granulation, extrusion/spheronization, roller-compaction, melt-congealing, and by spray-coating seed cores. See, for example, Multiparticulate Oral Drug Delivery; Ghebre-Sellassie Ed.; Marcel Dekker: 1994; and Pharmaceutical Pelletization Technology; Ghebre-Sellassie Ed.; Marcel Dekker: 1989.

Other excipients or carriers as described herein can be blended with the pharmaceutical composition to aid in processing and forming the multiparticulates. The resulting particles can themselves constitute the multiparticulate device or can be coated by various film-forming materials, such as enteric polymers, water-swellable, and water-soluble polymers. The multiparticulates can be further processed as a capsule or a tablet.

The compositions and formulations provided herein can also be formulated to be targeted to a particular tissue, receptor, or other area of the body of the subject to be treated, including liposome-, resealed erythrocyte-, and antibody-based delivery systems. Examples include, but are not limited to, those disclosed in U.S. Pat. Nos. 5,709,874; 5,759,542; 5,840,674; 5,900,252; 5,972,366; 5,985,307; 6,004,534; 6,039,975; 6,048,736; 6,060,082; 6,071,495; 6,120,751; 6,131,570; 6,139,865; 6,253,872; 6,271,359; 6,274,552; 6,316,652; and 7,169,410.

The compositions and formulations may also include carriers, adjuvants, excipients, preservatives, stabilizers, penetration enhancers, and the like.

The compositions can be in admixture with a suitable carrier, diluent, or excipient such as sterile water, physiological saline, glucose, or the like, and can contain auxiliary substances such as wetting or emulsifying agents, pH buffering agents, gelling or viscosity enhancing additives, preservatives, flavoring agents, colors, and the like, depending upon the route of administration and the preparation desired. See, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18th and 19th editions (December 1985, and June 1990, respectively). Such preparations can include complexing agents, metal ions, polymeric compounds such as polyacetic acid, polyglycolic acid, hydrogels, dextran, and the like, liposomes, microemulsions, micelles, unilamellar or multilamellar vesicles, erythrocyte ghosts or spheroblasts. Suitable lipids for liposomal formulation include, without limitation, monoglycerides, diglycerides, sulfatides, lysolecithin, phospholipids, saponin, bile acids, and the like. The presence of such additional components can influence the physical state, solubility, stability, rate of penetration, and rate of clearance, and are thus chosen according to the intended application, such that the characteristics of the carrier are tailored to the selected route of administration.

A pharmaceutically acceptable preservative can be employed to increase the shelf life of the compositions. Benzyl alcohol can be suitable, although a variety of preservatives including, for example, parabens, thimerosal, chlorobutanol, or benzalkonium chloride can also be employed. A suitable concentration of the preservative is typically from about 0.02% to about 2% based on the total weight of the composition, although larger or smaller amounts can be employed depending upon the agent selected. Reducing agents can be advantageously used to maintain acceptable shelf life of the formulations.

The compounds of various embodiments can be provided to an administering physician or other health care professional, or for self-administration by the patient, in the form of a kit. The kit is a package which houses a container which contains the compositions in suitable packaging, and instructions for administering the composition. The kit can optionally also contain one or more additional therapeutic agents. For example, a kit containing one or more topical compositions in combination with one or more additional anesthetic, antibacterial, and/or anti-inflammation agents can be provided. The kit can also contain separate doses for serial or sequential administration. The kit can optionally contain one or more diagnostic tools and instructions for use. The kit can contain suitable delivery devices, e.g., syringes, wipes, or the like, along with instructions for administering the compositions and any other agent. The kit can optionally contain instructions for storage, reconstitution (if applicable), and administration of any or all compositions included. The kits can include a plurality of containers reflecting the number of administrations to be given to a subject

### Methods of Use/Treatment

The compositions and formulation disclosed herein may be used to improve behavioral and social interaction in subjects diagnosed with neurodevelopmental disorders. In one embodiment, the compositions and formulations disclosed herein are used to treat subjects diagnosed with ASDs. In another embodiment, the compositions disclosed herein are used to treat subjects diagnosed with autism. In another embodiment, the compositions disclosed herein are used to treat subjects diagnosed with Asperger syndrome. In another embodiment, the compositions disclosed herein are used to treat subjects diagnosed with pervasive development disorder not otherwise specified. In another embodiment, the compositions disclosed herein are used to treat subjects diagnosed with childhood disintegrative disorder. In another embodiment, the compositions disclosed herein are used to treat subjects diagnosed with attention deficit hyperactivity disorder. In another embodiment, the compositions disclosed herein are used to treat subjects diagnosed with Tourette's syndrome. In another embodiment, the compositions disclosed herein are used to treat subjects diagnosed with schizophrenia. In another embodiment, the compositions disclosed herein are used to treat subjects diagnosed with epilepsy.

The compositions and formulations disclosed herein maybe used to treat symptoms associated autonomic nervous system disorders. In one embodiment, the compositions disclosed herein are used to treat subjects diagnosed with chronic fatigue syndrome. In another embodiment, the compositions disclosed herein are used to treat subjects diagnosed with fibromyalgia. In another embodiment, the compositions disclosed herein are used to treat subjects diagnosed with irritable bowel syndrome. In another embodiment, the compositions disclosed herein are used to treat subjects diagnosed with interstitial cystitis.

Treatment dosage may vary depending on the frequency (single or multiple doses), route of administration, severity of the condition being treated, sex, weight/body mass index, age, health, and dietary considerations. In one embodiment, the total daily dose is up to 1.5 g/kg of body weight. In another embodiment, the total daily dose is up to 1.25 g/kg of body weight. In another embodiment, the total daily dose is up to 1 g/kg of body weight. In another embodiment, the total daily dose is up to 750 mg/kg of body weight. In another embodiment, the total daily dose is up to 500 mg/kg body weight. In another embodiment, the total daily dose is up to 250 mg/kg of body weight. In another embodiment, the total daily dose is up to 100 mg/kg of body weight. In another embodiment, the total daily dose is up to 90 mg/kg of body weight. In another embodiment, the total daily dose is up to 80 mg/kg of body weight. In another embodiment, the total daily dose is up to 70 mg/kg of body weight. In another embodiment, the total daily dose is up to 60 mg/kg of body weight. In another embodiment, the total daily dose is up to 50 mg/kg of body weight. In another embodiment, the total daily dose is up to 40 mg/kg of body weight. In another embodiment, the total daily dose is up to 30 mg/kg of body weight. In another embodiment, the total daily dose is up to 20 mg/kg of body weight. In another embodiment, the total daily dose is up to 10 mg/kg of body weight. In another embodiment, the total daily dose is up to 5 mg/kg of body weight. In another embodiment, the total daily dose is up to 3 mg/kg of body weight. In another embodiment, the total daily dose is up to 1 mg/kg of body weight.

In one embodiment, the total daily dose is given in a single dose. In another embodiment, the total daily dose is given in two doses. In this embodiment, the two doses may be given in succession, such as one dose in each nostril or one dose in each eye or two puffs on an inhaler. Alternatively, the two doses may be administered at different times, such as at least 5 minutes between doses, at least 10 minutes between doses, at least 15 minutes between doses, at least 20 minutes between doses, at least 30 minutes between doses, at least 45 minutes between doses, at least 1 hour between doses, at least 2 hours between doses, at least 3 hours between doses, at least 4 hours between doses, at least 5 hours between doses, at least 6 hours between doses, at least 7 hours between doses, at least 8 hours between doses, at least 9 hours between doses, at least 10 hours between doses, at least 11 hours between doses, at least 12 hours between doses, at least 13 hours between doses, at least 14 hours between doses, at least 15 hours between doses, at least 16 hours between doses, at least 17 hours between doses, at least 18 hours between doses, at least 19 hours between doses, at least 20 hours between doses, at least 21 hours between doses, at least 22 hours between doses, or at least 23 hours between doses. In another embodiment, the total daily dose is given in three doses, either in succession, timed delay (e.g. every few minutes or hour(s)), or spread throughout the day. In another embodiment, the total daily dose is given in four doses, either in succession, timed delay (e.g. every few minutes or hour(s)), or spread throughout the day. In another embodiment, the total daily dose is given in five doses, either in succession, timed delay (e.g. every few minutes or hour(s)), or spread throughout the day. In another embodiment, the total daily dose is given in six doses, either in succession, timed delay (e.g. every few minutes or hour(s)), or spread throughout the day. In another embodiment, the total daily dose is given in seven doses, either in succession, timed delay (e.g. every few minutes or hour(s)), or spread throughout the day. In another embodiment, the total daily dose is given in eight doses, either in succession, timed delay (e.g. every few minutes or hour(s)), or spread throughout the day.

### EXAMPLES

### Example 1—

In this example the safety and efficacy of a single dose of suramin was evaluated in children diagnosed with autism spectral disorder. Eligible study participants were males between the ages of 4 to 17 years diagnosed with autism and with an autism diagnostic observation schedule (ADOS) scores of at least 7 and free of prescription medications and other medical issues such as seizures, heart, liver, kidney, or adrenal disease.

Study participants were randomly divided into two groups. The first group were given 20 mg/kg of suramin in 50 ml of saline by intravenous infusion over 30 minutes. The second group were given 50 ml of saline by intravenous infusion over 30 minutes.

Study participants were evaluated over a 6 week period following the single dose of suramin. Primary outcome measures were (1) joint social attention as measured by the ADOS reciprocal social interaction domain scores and (2) receptive language as measured by the Peabody picture vocabulary test (PPVT). Secondary outcome measures were (1) social interaction as measured by blinded examiner scoring of video recorded ADOS evaluations using elements of the Communication and Symbolic Behavior Scales (CSBS); (2) expressive language as measured by the expressive one word picture vocabulary test (EOWPVT); (3) cortical connectivity as measured by EEG power and coherence analysis; (4) autonomic nervous system balance as measured by EKG analysis of heart rate variability; (5) praxia and motor coordination as measured by fine motor and gross motor digital balance board and dynamic gait analysis; (6) aberrant behaviors as measured by aberrant behavior checklist (ABC); (7) clinical global impression—improvement scale (CGI-I) as measured by the 7-point scale reflecting the change in core autism behaviors; (8) global assessment of child development behaviors as measured by child behavior checklist (CBCL); and (9) repetitive behaviors as measure by the repetitive behavior questionnaire (RBQ).

### Example 2—

In this example, the safety and efficacy of single dose of suramin administered intranasally is compared to a single dose of suramin administered intravenously. Dosing and study participant requirements are as described in Example 1. Study participants are randomly assigned to receive either the intranasal formulation or the intravenous formulation. Due to the obvious difference in delivery methods, participants and medical personnel administering the drug are not blinded. Post dose evaluations are as described in Example 1. Additionally, study participants will be evaluated to see how long the effects of the drug last.

### Example 3—

In this example, the safety and efficacy of single dose of suramin administered as an inhalant is compared to a single dose of suramin administered intravenously. Dosing and study participant requirements are as described in Example 1. Study participants are randomly assigned to receive either the inhalant formulation or the intravenous formulation. Due to the obvious difference in delivery methods, participants and medical personnel administering the drug are not blinded. Post dose evaluations are as described in Example 1. Additionally, study participants will be evaluated to see how long the effects of the drug last.

### Example 4—

In this example, the safety and efficacy of single dose of suramin administered sublingually is compared to a single dose of suramin administered intravenously. Dosing and study participant requirements are as described in Example 1. Study participants are randomly assigned to receive either the sublingual formulation or the intravenous formulation. Due to the obvious difference in delivery methods, participants and medical personnel administering the drug are not blinded. Post dose evaluations are as described in Example 1. Additionally, study participants will be evaluated to see how long the effects of the drug last.

### Example 5—

In this example, the safety and efficacy of multiple doses of suramin are evaluated in children diagnosed with autism spectral disorder. Study participant requirements are as described in Example 1. Study participants are randomly assigned to three different dosing schedules. In the first dosing schedule, the study participants receive three separate doses of suramin at 20 mg/kg or body weight every two weeks. In the second dosing schedule, the study participants receives three separate doses of suramin at 20 mg/kg or body weight every month. In the fourth dosing schedule, the study participants receive three separate doses of suramin at 20 mg/kg or body with each dose given when at least half of the primary and secondary outcome behaviors described in Example 1 begin to regress. Post dose evaluations are as described in Example 1. Additionally, study participants will be evaluated to see how long the effects of the last dose lasts.

### Example 6—

In this example, the safety and efficacy of alternate dosing amounts are evaluated in children diagnosed with autism spectral disorder. Study participant requirements are as described in Example 1. Study participants are randomly assigned to three different dose amounts. In the first group, the study participants receive a single dose of suramin at 10 mg/kg of body weight. In the second group, the study participants receive a single dose of suramin at 20 mg/kg of body weight. In the third group, the study participants receive a single dose of suramin at 40 mg/kg of body weight. Post dose evaluations are as described in Example 1. Additionally, study participants will be evaluated to see how long the effects of the drug last.

The examples set forth above are provided to give those of ordinary skill in the art a complete disclosure and description of how to make and use embodiments of the compositions, and are not intended to limit the scope of what the inventors regard as their invention. Modifications of the above-described modes (for carrying out the invention that are obvious to persons of skill in the art) are intended to be within the scope of the following claims. All publications, patents and patent applications cited in this specification are incorporated herein by reference as if each such publication, patent or patent application were specifically and individually indicated to be incorporated herein by reference.

1. A composition for use in treating neurodevelopmental disorders comprising:
an antipurinergic compound; and
a carrier;
wherein the antipurinergic compound is formulated for non-intravenous administration.

2. The composition of claim 1, wherein the antipurinergic compound is suramin.

3. The composition of claim 1, wherein the neurodevelopmental disorder is selected from the group consisting of autism spectral disorders, attention deficit hyperactivity disorder, Tourette's syndrome, schizophrenia, and epilepsy.

4. The composition of claim 3, wherein the autism spectral disorder is selected from the group consisting of autism, Asperger's syndrome, pervasive developmental disorder not otherwise specified, and childhood disintegrative disorder.

5. The composition of claim 4, wherein the autism spectral disorder is autism.

6. The composition of claim 1, wherein the non-intravenous delivery is selected from the group consisting of: intranasal, inhalant, sublingual, buccal, lingual, gingival, transdermal, ocular, vaginal, urethral, rectal, and implantable devices.

7. The composition of claim 6, wherein the non-intravenous administration is sublingual.

8. The composition of claim 6, wherein the non-intravenous delivery is intranasal.

9. The composition of claim 6, wherein the non-intravenous delivery is inhalant.

10. The composition of claim 6, wherein the non-invasive delivery is an implantable device.

11. The composition of claim 1, further comprising a stabilizer.

12. The composition of claim 1, further comprising a preservative.

13. The composition of claim 1, further comprising a penetration enhancer.

14. The composition of claim 1, further comprising an extended release agent.

15. A method for treating a neurodevelopmental disorder in a subject comprising the step of administering to the subject the composition of claim 1.

16. The method of claim 15, wherein the neurodevelopmental disorder is selected from the group consisting of autism spectral disorders, attention deficit hyperactivity disorder, Tourette's syndrome, schizophrenia, and epilepsy.

17. The method of claim 16, wherein the autism spectral disorder is selected from the group consisting of autism, Asperger's syndrome, pervasive developmental disorder not otherwise specified, and childhood disintegrative disorder.

18. The method of claim 15, wherein the non-intravenous delivery is selected from the group consisting of: intranasal, inhalant, sublingual, buccal, lingual, gingival, transdermal, ocular, vaginal, urethral, rectal, and implantable devices.
